SEQUENCE LISTING (6 sequences)

SEQ ID NO: 1 - DNA, 20 bp, Artificial Sequence
  an artificially synthesized primer sequence
aaaagccctgccctgttgct

SEQ ID NO: 2 - DNA, 20 bp, Artificial Sequence
  an artificially synthesized primer sequence
ccccgaacaagaaggtcccc

SEQ ID NO: 3 - DNA, 25 bp, Artificial Sequence
  an artificially synthesized probe sequence
tgctgtgtttactctcccgtgtgcc

SEQ ID NO: 4 - DNA, 22 bp, Artificial Sequence
  an artificially synthesized primer sequence
cgacttgtgaacgagttgttgg

SEQ ID NO: 5 - DNA, 22 bp, Artificial Sequence
  an artificially synthesized probe sequence
ttcagtgagacagaggcaggtg

SEQ ID NO: 6 - DNA, 28 bp, Artificial Sequence
  an artificially synthesized primer sequence
tgttgtaactgcccaaggcatgttttgc

(19) United States
(12) Patent Application Publication
(10) Pub. No.: US 2003/0148312 A1
(43) Pub. Date: Aug. 7, 2003

(54) Method for testing for allergic disease

(75) Inventors: Nei Yoshida, Kanagawa (JP); Naoko Nagata, Kanagawa (JP); Tadahiro Oshida, Kanagawa (JP); Ryoichi Hashida, Kanagawa (JP); Yuji Sugita, Kanagawa (JP); Hirohisa Saito, Tokyo (JP)

Correspondence Address: LAHIVE & COCKFIELD, 28 STATE STREET, BOSTON, MA, 02109, US

(73) Assignee: NATIONAL CENTER FOR CHILD HEALTH AND DEVELOPMENT, Tokyo (JP)

(21) Appl. No.: 10/205,298

(22) Filed: Jul. 24, 2002

(30) Foreign Application Priority Data
Jul. 24, 2001 (JP) 2001-222923 JP

(51) Int. Cl. C12Q 1/68; G01N 33/53; G01N 33/567; C07H 21/04

(52) U.S. Cl. 435/6; 435/7.2; 536/24.3

(57) ABSTRACT

An objective of the present invention is to provide a method for testing for an allergic disease and a method of screening for a therapeutic agent for allergic diseases.

MAL was identified as a gene the expression level of which is significantly increased in T cells contained in the peripheral blood monocyte (PBMC) upon stimulation thereof with a mite allergen in vitro. The present inventors found that this gene can be used in testing for allergic diseases and in screening for agents and compounds useful in the treatment of allergic diseases.

### FIELD OF THE INVENTION

The present invention relates to a method for testing for an allergic disease.

### BACKGROUND OF THE INVENTION

Allergic diseases are considered to be multifactorial diseases. In other words, bronchial asthma and atopic dermatitis are caused by the interaction of many different genes, each of which is influenced by various environmental factors. Thus, it has been extremely difficult to identify a specific gene which causes a allergic disease.

The expression of mutated or defective genes, or overexpression or reduction of the expression of specific gene is thought to be involved in allergic diseases. To elucidate the role of gene expression in diseases, it is necessary to understand how a gene is involved in triggering disease onset and how expression of the gene is altered by external stimulants such as drugs.

In recent diagnosis of allergic diseases, history taking, and confirmation of the patient's family history and own anamnesis are important factors in general. In addition, for diagnosis of allergy based on more objective information, a test method using patient's blood sample and method for observing patient's immune response to allergen are also performed. Examples of the former method are the allergen-specific IgE measurement, leukocyte histamine release test, lymphocyte stimulating test, or the like. Presence of an allergen-specific IgE is a proof for the allergic reaction to that allergen. However, in some patients, allergen-specific IgE may not necessarily be detected. Furthermore, the assay principle of IgE requires performing tests for all of the allergens necessary for diagnosis. Leukocyte histamine release test and lymphocyte stimulating test are the methods for observing the immune system reaction toward a specific allergen in vitro. These methods are complicate in operation.

On the other hand, another method is also known, wherein the immune response observed when a patient is actually contacted with an allergen is used for diagnosing an allergy (latter method). Such a test includes the prick test, scratch test, patch test, intradermal reaction, or induction test. Indeed these tests allow the direct diagnosis of patient's allergic reaction but they can be said to be highly invasive tests wherein patients are actually exposed to allergen.

In addition, regardless of the allergen types, test methods for proving the involvement of allergic reaction are also attempted. For example, a high serum IgE titer may indicate the occurrence of allergic reaction in the patient. The serum IgE titer is information corresponding to the total amount of allergen-specific IgE. Though it is easy to determine the total amount of IgE regardless of the type of allergen, IgE titer may be reduced in some patients with a non-atopic bronchitis or the like.

Therefore, a marker (indicator) for an allergic disease that is not only less invasive to patients but also capable of readily providing information necessary for diagnosis would be useful. Since such markers are thought to be profoundly involved in triggering disease onset, they may become the important target in not only diagnosis but also control of allergic symptoms.

### SUMMARY OF THE INVENTION

An objective of the present invention is to provide an indicator gene enabling the test for allergic disease, in particular. Another objective of the invention is to provide a method for testing for an allergic disease and a method of screening for a therapeutic agent for an allergic disease based on the indicator gene.

A variety of blood cells are closely associated with immune response. For example, the immune response to the mite antigen proceeds as follows. First, monocytes phagocyte the mite antigen, and present digests thereof as the antigen to T cells. Only T cells capable of recognizing the presented peptide of mite antigen respond to the antigen presentation, producing a variety of cytokines that determine the direction of the subsequent immune reaction (allergic reaction). Cytokines produced at this time are those such as IL-4, IL-5, IFN-γ, and so forth, which closely associated with allergic reactions. As a result of such activities of T cells, the production of mite antigen-specific IgE is initiated in B cells by the interaction thereof with T cells and stimulation by IL-4.

The present inventors thought it possible to isolate an allergic reaction-associated gene by observing alterations in the gene expression in blood cells supporting such an immune response system. Based on such a concept, the present applicants succeeded in isolating the following genes, the expression levels of which alter in blood cells of peripheral blood from patients with pollinosis, and filed a patent application:

pollinosis-associated gene 373 (WO 00/65046),

pollinosis-associated gene 419 (WO 00/65045),

pollinosis-associated gene 513 (WO 00/65049),

pollinosis-associated gene 581 (WO 00/65048),

pollinosis-associated gene 795 (WO 00/65050),

pollinosis-associated gene 627 (WO 00/65051),

pollinosis-associated gene 441 (WO 00/73435),

pollinosis-associated gene 465 (WO 00/73439), and

pollinosis-associated gene 787 (WO 00/73440).

These genes are those showing differential expression levels in cells freshly isolated from peripheral blood. This type of gene expression is called a spontaneous expression. For such an approach to the problem, the present inventors have actively studied so as to place cells separated from peripheral blood in condition similar to the affected part of allergic disease patient.

On the other hand, a technique to observe the allergic reaction in vitro has been known, wherein the peripheral blood mononuclear cell (hereafter abbreviated PBMC) is stimulated with an allergen. The present inventors further improved this technique to search for genes, the expression levels of which alter in T cells, in particular, following the treatment with an allergen. As described above, T cells are the cells determining the immune response. Therefore, at the time of occurrence of allergic reaction toward an allergen, a gene changes its expression level in T cells, which is assumed to play an important role in that reaction.

Through such an analysis, the present inventors proved that the expression level of MAL gene is significantly elevated together with the allergic immune response. Based on these information, the present inventors found it possible to test for an allergic disease or screen for therapeutic agent for allergic disease using the MAL gene as an indicator, accomplishing the present invention. That is, the present invention relates to the following method for testing for allergy, therapeutic agent for an allergic disease, method of screening for that therapeutic agent, model animal for allergic diseases, and kit for performing these methods:

[1] a method for testing for an allergic disease using MAL as an indicator gene, said method comprising the steps of:

(a) measuring the expression level of the indicator gene in a biological sample from a subject, and

(b) comparing the expression level measured in (a) with that in a biological sample from a normal healthy subject;

[2] the method according to [1], wherein the allergic disease is atopic dermatitis and/or bronchial asthma;

[3] the method according to [1], wherein the expression level of the gene is measured by PCR of the cDNA for the gene;

[4] the method according to [1], wherein the expression level of the gene is measured by detecting a protein encoded by said gene;

[5] the method according to [1], wherein the biological sample is a sample comprising peripheral blood T cells;

[6] the method according to [1], wherein the expression level of the indicator gene is measured after peripheral blood mononuclear cells (PBMC) are stimulated with an allergen;

[7] a reagent for testing for allergic disease, said reagent comprising an oligonucleotide that comprises a nucleotide sequence complementary to a polynucleotide comprising the nucleotide sequence of MAL gene or to the complementary strand thereof and that comprises at least 15 nucleotides;

[8] a reagent for testing for allergic disease, said reagent comprising an antibody that recognizes a peptide comprising the amino acid sequence encoded by MAL gene;

[9] the reagent according to [7] or [8], said reagent further comprising an allergen;

[10] a method of screening for a therapeutic agent for an allergic disease using MAL gene or a gene functionally equivalent thereto as an indicator gene, said method comprising the steps of:

(a) contacting a candidate compound with a cell expressing the indicator gene,

(b) measuring the expression level of the indicator gene, and

(c) selecting a compound that reduces the expression level of the indicator gene, compared to a control;

[11] the method according to [10], wherein the cell is T cell;

[12] a method of screening for a therapeutic agent for an allergic disease using MAL gene or a gene functionally equivalent thereto as an indicator gene, said method comprising the steps of:

(a) administering a candidate compound to a test animal,

(b) measuring the expression level of the indicator gene in a biological sample from the test animal, and

(c) selecting a compound that reduces the expression level of the indicator gene, compared to a control;

[13] the method according to [12], said method comprising the step of stimulating the test animal with an allergen before or after step (a);

[14] a method of screening for a therapeutic agent for an allergic disease using MAL gene or a gene functionally equivalent thereto as an indicator gene, said method comprising the steps of:

(a) contacting a candidate compound with a cell into which a vector comprising the transcriptional regulatory region of the indicator gene and a reporter gene that is expressed under the control of the transcriptional regulatory region has been introduced,

(b) measuring the activity of said reporter gene, and

(c) selecting a compound that reduces the expression level of said reporter gene, compared to a control;

[15] a method of screening for a therapeutic agent for an allergic disease using MAL gene or a gene functionally equivalent thereto as an indicator gene, said method comprising the steps of:

(a) contacting a candidate compound with a protein encoded by the indicator gene,

(b) measuring the activity of said protein, and

(c) selecting a compound that reduces the activity of said protein, compared to a control;

[16] a therapeutic agent for an allergic disease, said agent comprising, as an active ingredient, a compound that is obtained by the method according to any one of [10], [12], [14], and [15];

[17] a therapeutic agent for an allergic disease, said agent comprising an antisense DNA against an indicator gene or a portion thereof as a principal ingredient, wherein the indicator gene is MAL gene or a gene functionally equivalent to MAL gene;

[18] a therapeutic agent for an allergic disease, said agent comprising, as a principal ingredient, an antibody that binds to a protein encoded by an indicator gene, wherein the indicator gene is MAL gene or a gene functionally equivalent to MAL gene;

[19] a method for producing an allergic disease model animal using MAL gene or a gene functionally equivalent thereto as an indicator gene, said method comprising a step of elevating expression level of the indicator gene in T cells of a non-human vertebrate;

[20] a kit for screening for a therapeutic agent for an allergic disease, said kit comprising

an oligonucleotide that comprises a nucleotide sequence complementary to a polynucleotide comprising a nucleotide sequence of an indicator gene or to the complementary strand thereof and that comprises at least 15 nucleotides and

cells expressing the indicator gene,

wherein the indicator gene is the MAL gene or a gene functionally equivalent to MAL gene;

[21] a kit for screening for a therapeutic agent for an allergic disease, said kit comprising an antibody that recognizes a peptide comprising an amino acid sequence encoded by an indicator gene and cells expressing the indicator gene, wherein the indicator gene is MAL gene or a gene functionally equivalent to MAL gene; and

[22] the kit according to [20] or [21], said kit further comprising an allergen.

In addition, this invention relates to a method for treating an allergic disease, the method comprising the step of administering any one of compounds described below. This invention also relates to the use of any one of compounds described below for the manufacture of a therapeutic agent for an allergic disease:

a compound that can be obtained by the screening method according to any one of [10], [12], [14], and [15];

an antisense DNA against MAL gene, a gene functionally equivalent to MAL gene, or a portion thereof; and

an antibody that binds to a protein encoded by MAL gene.

The structure of the MAL gene serving as an indicator gene in the present invention has been already revealed. The MAL gene cloned from T cells is expressed in the metaphase and anaphase of the T-cell differentiation, and assumed to play a certain role in that differentiation (Miguel A. Alonso and Sherman M. Weissman (1987) cDNA cloning and sequence of MAL, a hydrophobic protein associated with human T-cell differentiation. Proc. Natl. Acad. Sci. USA 84: 1997-2001; Carmen Rancano, Teresa Rubio, et al. (1994) Alternative splicing of human T cell-specific MAL mRNA and its correlation with the exon/intron organization of the gene. Genomics. 21: 447-450). In humans, MAL has been reported to be expressed in, as well as T cell, the central nervous system, gray matter of the cerebral cortex, and thyroid gland (Jane A. Wakeman, Paul R. Heath, et al. (1997) MAL mRNA is induced during the differentiation of human embryonal carcinoma cells into neurons and is also localized within specific regions of human brain. Differentiation. 62: 97-105; Fernando Martin-Belmonte, Leonor Kremer, et al. (1998) Expression of the MAL gene in the thyroid: the MAL proteolipid, a component of glycolipid-enriched membranes, is apically distributed in thyroid follicles. Endocrinology. 139: 2077-2084). MAL, as a component of membrane vesicle involved in the protein transport, is thought to participate in the sorting of proteins, transport thereof to membranes, and construction, stabilization, and maintenance of the membrane microstructure (Marcus Frank. (2000) MAL, a proteolipid in glycosphingolipid enriched domains: functional implication in myelin and beyond. Progress in Neurobiology 60: 531-544).

Also, it has been reported that MAL may be involved in the transport of myelin protein to the myelin membrane in the central nervous system, and delivery of various proteins including the glycosylphosphatidylinositol (GPI)-anchored protein to the apical membrane in the epithelial cell (Fernand Martin-Belmonte, Rosa Puertollano et al. (2000) The MAL Proteolipid is Necessary for the Overall Apical Delivery of Membrane Proteins in the Polarized Epithelial Madin-Darby Canine Kidney and Fisher Rat Thyroid Cell Lines. Molecular Biology of the Cell 11: 2033-2045).

The MAL protein is present in the glycolipid-enriched membrane (GEM) microdomains of human T-cell membrane. Participation of GEM in the signal transduction and, furthermore, the coprecipitation of CD59 and tyrosine kinase Lck involved in the signal transduction with anti-MAL antibody indicate a possibility that MAL is also involved in the signal transduction (Jaime Millan, Rosa Puertollano, et al. (1997). The MAL proteolipid is a component of the detergent-insoluble membrane subdomains of human T-lymphocytes. Biochem. J. 321: 247-252; Jaime Millan and Miguel A. Alonso (1998). MAL, a novel integral membrane protein of human T lymphocytes, associates with glycosylphosphatidylinositol-anchored proteins and Src-like tyrosine kinases. Eur. J. Immunol. 28: 3675-3684). In addition, there is a report (WO 88/07549) indicating the association of MAL with the T-cell activation, however, without presenting the basis for the association. Furthermore, another report indicated the association of MAL with cancer (WO 97/33551).

However, no report has so far described a specific function of MAL including the involvement thereof in the signal transduction in T cell. Furthermore, there is no report at all indicating the association of MAL with the immune reaction or allergic reaction, and the differentiation of T cells into Th2 cells essential for the establishment of allergic reaction.

### BRIEF DESCRIPTION OF THE DRAWINGS

FIG. 1 shows changes in the MAL expression level in T cells following the mite antigen stimulation. In this figure, the longitudinal axis represents Average Difference (Avg Diff), the average of difference in fluorescence intensity between perfect-matched and mismatched probe cells, and the horizontal axis shows samples and incubation time. C: the unstimulated control; M: cells stimulated with the mite antigen; D: cells treated with the mite antigen and dexamethasone.

FIG. 2 shows changes in the MAL expression level in the mite antigen stimulation. The copy number of MAL mRNA per 1 ng of RNA in PBMC is indicated for each sample. N: normal healthy subject; P: patient; Score: mite-specific IgE score.

FIG. 3 shows changes in the IL-4 receptor α chain expression level in the mite antigen stimulation. The copy number of mRNA of the IL-4 receptor α chain per 1 ng of RNA in PBMC is indicated for each sample. N: normal healthy subject; P: patient; Score: mite-specific IgE score.

FIG. 4 shows the results of measurement of the IL-4 concentration in the culture supernatant of PBMC stimulated with the mite antigen. The IL-4 concentration (pg/ml) in the culture solution is indicated for each sample. N: normal healthy subject; P: patient; Score: mite-specific IgE score.

FIG. 5 shows the results of comparison of the MAL expression level in various leukocytes of the peripheral blood. The longitudinal axis represents the copy number of MAL mRNA per 1 ng of RNA, and the horizontal axis shows the types of leukocytes.

FIG. 6 shows the increase of MAL in the cultured peripheral blood T cells by the IL-4 stimulation. The longitudinal axis represents the copy number of MAL mRNA per 1 ng of the RNA, and the horizontal axis shows the cytokine stimulation time.

FIG. 7 is a photograph that shows the results of Western blotting of the MAL protein expression level in Jurkat cells stimulated with IL-4. Anti-LAT antibody (upper) or anti-MAL antibody (lower) was used, and, for each antibody, the results were compared with those in the absence of IL-4 stimulation.

FIG. 8 is a photograph that shows the results of Western blotting of the MAL protein expression level in peripheral blood cultured T-cells stimulated with IL-4. Anti-LAT antibody (upper) or anti-MAL antibody (lower) was used, and, for each antibody, the results were compared with those in the absence of IL-4 stimulation.

### DETAILED DESCRIPTION OF THE INVENTION

In the present invention, allergic disease is a general term for diseases in which allergic reactions is involved. More specifically, for a disease to be considered allergic, the allergen must be identified, a strong correlation between exposure to the allergen and the onset of the pathological change must be demonstrated, and the pathological change has been proven to have an immunological mechanism. Herein, an immunological mechanism means that the leukocytes show an immune response to allergen stimulation. Examples of allergens are the mite antigen, pollen antigen, etc. Representative allergic diseases are bronchial asthma, allergic rhinitis, pollen allergy, insect allergy, etc. Allergic diathesis is a genetic factor that is inherited from allergic parents to children. Familial allergic diseases are also called atopic diseases, and their causative factor that can be inherited is atopic diathesis. Among atopic diseases, asthma is a general term for diseases accompanied with respiratory organ symptoms.

A method for testing for an allergic disease in the present invention comprises the steps of measuring the expression level of the MAL gene in biological samples from a subject, and comparing the measured value with that of a normal healthy subject. As a result of comparing both values, when the MAL gene expression is enhanced compared to that in a normal healthy subject, the subject may be diagnosed with an allergic disease. In the present invention, the MAL gene that can serve as an indicator for an allergic disease is called an indicator gene.

Indicator gene is not limited to only the MAL gene, which may be combined with a gene that can be an indicator for other allergic disease. Measurement using a combination of a plurality of genes as the indicator may improve the testing accuracy. Since patients with allergic disease such as bronchial asthma are a heterogeneous population, they may be more accurately diagnosed by using a plurality of genes as an indicator for allergy.

In the present invention, the expression level of an indicator gene includes the transcription of the gene to mRNA as well as the translation thereof into a protein. Therefore, a method for testing for an allergic disease according to the present invention is performed based on the comparison of expression intensity of mRNA corresponding to the indicator gene, or expression level of a protein encoded by the indicator gene.

Measurement of the expression level of an indicator gene in a test for an allergic disease in the present invention can be performed according to the known gene analytical method. More specifically, for example, a hybridization technique with a nucleic acid as a probe that hybridizes to this gene, a gene amplification technique with a DNA hybridizing to the gene of this invention as a primer, or the like can be utilized.

The probe or primer used in the test of the present invention can be designed based on the nucleotide sequence of the indicator gene. For example, the nucleotide sequence of the human MAL gene is known as GenBank Acc. No. X76223.

Genes of higher animals are generally accompanied by polymorphism in a high frequency. There exist many molecules that produce isoforms comprising different amino acid sequences from each other during the splicing process. Any genes associated with allergy which have a similar activity to that of the indicator gene are included in the indicator gene of the present invention, even though they carry mutation in the nucleotide sequence due to polymorphism and isoform.

As a primer or probe can be used a polynucleotide comprising the nucleotide sequence of the indicator gene or at least 15 nucleotides that are complementary to the complementary strand thereof. Herein, the term “complementary strand” means one strand of a double stranded DNA composed of A:T (U for RNA) and G:C base pairs to the other strand. In addition, “complementary” means not only those completely complementary to a region of at least 15 continuous nucleotides, but also having a homology of at least 70%, preferably at least 80%, more preferably 90%, and even more preferably 95% or higher. The degree of homology between nucleotide sequences can be determined by the algorithm, BLAST, etc.

Such polynucleotides are useful as the probe to detect an indicator gene, or as the primer to amplify the indicator gene. When used as a primer, those polynucleotides comprises usually 15 bp˜100 bp, preferably 15 bp˜35 bp of nucleotides. When used as a probe, DNAs comprising the whole sequence of the indicator gene (or a complementary strand thereof), or a partial sequence thereof that contains at least 15-bp nucleotides. When used as a primer, the 3′ region thereof must be complementary to the indicator gene, while the 5′ region can be linked to a restriction enzyme-recognition sequence or tag.

“Polynucleotides” in the present invention may be either DNA or RNA. These polynucleotides may be either synthetic or naturally-occurring. Also, DNA used as a probe for hybridization is usually labeled. Examples of labeling methods are those as described below. Herein, the term “oligonucleotide” means a polynucleotide with relatively low degree of polymerization. Oligonucleotides are included in polynucleotides. The labeling methods are as follows:

nick translation labeling using DNA polymerase I;

end labeling using polynucleotide kinase;

fill-in end labeling using Klenow fragment (Berger, S L, Kimmel, Ark. (1987) Guide to Molecular Cloning Techniques, Method in Enzymology, Academic Press; Hames, B D, Higgins, S J (1985) Genes Probes: A Practical Approach. IRL Press; Sambrook, J, Fritsch, E F, Maniatis, T. (1989) Molecular Cloning: a Laboratory Manual, 2nd Edn. Cold Spring Harbor Laboratory Press);

transcription labeling using RNA polymerase (Melton, D A, Krieg, P A, Rebagkiati, M R, Maniatis, T, Zinn, K, Green, M R. (1984) Nucleic Acid Res., 12, 7035-7056); and

non-isotopic labeling of DNA by incorporating modified nucleotides (Kricka, L J. (1992) Nonisotopic DNA Probing Techniques. Academic Press).

For testing for an allergic disease using hybridization techniques, for example, Northern hybridization, dot blot hybridization, or DNA microarray technique may be used. Furthermore, gene amplification techniques, such as RT-PCR method may be used. By using the PCR amplification monitoring method during the gene amplification step in RT-PCR, one can achieve more quantitative analysis for the gene expression of the present invention.

In the PCR gene amplification monitoring method, the detection target (DNA or reverse transcript of RNA) is hybridized to probes that are dual-labeled at both ends with different fluorescent dyes whose fluorescences cancel each other out. When the PCR proceeds and Taq polymerase degrades the probe with its 5′-3′ exonuclease activity, the two fluorescent dyes become distant from each other and the fluorescence becomes to be detected. The fluorescence is detected in real time. By simultaneously measuring a standard sample in which the copy number of the target is known, it is possible to determine the copy number of the target in the subject sample with the cycle number where PCR amplification is linear (Holland, P. M. et al., 1991, Proc. Natl. Acad. Sci. USA 88: 7276-7280; Livak, K. J. et al., 1995, PCR Methods and Applications 4(6): 357-362; Heid, C. A. et al., 1996, Genome Research 6: 986-994; Gibson, E. M. U. et al., 1996, Genome Research 6: 995-1001). For the PCR amplification monitoring method, for example, ABI PRISM7700 (PE Biosystems) may be used.

The method for testing for an allergic disease in the present invention can be also carried out by detecting a protein encoded by the indicator gene. Hereinafter, a protein encoded by the indicator gene is described as an indicator protein. For such test methods, for example, Western blotting method, immunoprecipitation method, and ELISA method may be employed using antibody that binds to the indicator protein.

Antibodies that bind to the indicator protein used in the detection may be produced by techniques known to those skilled in the art. Antibodies used in the present invention may be polyclonal or monoclonal antibodies (Milstein, C. et al., 1983, Nature 305 (5934): 537-40). For example, polyclonal antibody against an indicator protein may be produced by collecting the blood from mammals sensitized with the antigen, and separating the serum from this blood using known methods. As a polyclonal antibody, the serum containing polyclonal antibody as such may be used. As the occasion demands, a fraction containing polyclonal antibody can be further isolated from this serum. Also, monoclonal antibody may be obtained by isolating immune cells from mammals sensitized with the antigen, fusing these cells with myeloma cells, and such, cloning hybridomas thus obtained, and collecting the antibody as a monoclonal antibody from the culture of the hybridomas.

For detecting an indicator protein, these antibodies may be appropriately labeled. Alternatively, instead of labeling the antibody, a substance that specifically binds to the antibody, for example, protein A or protein G, may be labeled to arrange an indirect detection of indicator protein. More specifically, one example of an indirect detection method is ELISA.

Protein or its partial peptide used as an antigen may be obtained, for example, by inserting the gene or its portion into an expression vector, introducing it into an appropriate host cell to produce a transformant, culturing the transformant to express the recombinant protein, and purifying the expressed recombinant protein from the culture or the culture supernatant. Alternatively, amino acid sequences encoded by these genes, or oligopeptides comprising portions of the amino acid sequence encoded by the full-length cDNA are chemically synthesized to be used as the antigen.

Furthermore, in the present invention, a testing for an allergic disease can be performed using not only the expression level of an indicator gene but also the activity of an indicator protein in the biological sample as an index. Activity of an indicator protein means a biological activity intrinsic to each protein. The detection of activity of an indicator protein can be achieved by known method.

In a test method of the present invention, usually a biological sample from a subject is used as a test sample, including peripheral blood T cells, etc. T cell is collected from peripheral blood using a known method. That is, PBMC can be separated by centrifuging the diluted peripheral blood with Ficoll. The test method of the present invention can be performed by measuring the amount of indicator gene or indicator protein using the isolated PBMC as a test sample. PBMC comprises lymphocytes and monocytes. However, as shown in Examples, a high-level expression of the indicator gene is observed in T cells among these blood cells. Therefore, the co-presence of other type cells exerts almost no influence on the results of indicator gene and indicator protein measurements. Alternatively, T cells can be separated by specifically adsorbing to microbeads on which the anti-CD3 antibody is immobilized.

Furthermore, as the biological sample in the present invention, blood, sputum, secretion from nasal mucosa, bronchoalveolar lavage fluid, lung scrape, and such may be used. These biological samples are collected using known methods. When PBMC or the whole blood is used as the test sample, blood cells are disintegrated to measure the indicator gene mRNA or indicator protein in the cell.

In the present invention, measurement of the indicator gene can be performed after PBMC is stimulated by an allergen. By measuring levels of the protein production and gene transcription induced by the indicator gene in T cells contained in PBMC after its stimulation with an allergen, it is possible to judge whether an allergic immune response actually occurs, and, furthermore, determine the response intensity. Thus, it enables the confirmation of added antigen as an allergen, and prediction of severity of disease.

In the present invention, the known allergic substances may be used as the allergen. More specifically, mites, house dust, plant pollen, or proteins originating in various foods are known as the allergic substances. These allergens may be either naturally occurring ones, or synthetic ones produced by gene recombination techniques, etc. Furthermore, allergens can be fragments of these proteins. Methods for preparing purified allergens are also known.

A method for stimulating PBMC by an allergen in vitro is known. For example, as described in Examples, the isolated PBMC and such can be stimulated by adding an allergen thereto. Stimulation by an allergen is transmitted to the antigen-recognition T cells via phagocytosis and antigen presentation by monocytes, initiating the immune response.

The measurement value of expression level of the indicator gene in T cells can be revised by a known method, so that changes in the gene expression levels in cells can be compared each other. The revise of measurement values is carried out by revising the measurement value of the expression level of a gene to be used as an index in this invention based on the measurement value of the expression level of a gene (house-keeping gene) the expression level of which in T cells is not widely altered regardless of the cellular conditions.

Furthermore, the present invention provides a reagent for the testing method of this invention. That is, this invention relates to a reagent for testing for bronchial asthma, said reagent comprising an oligonucleotide that comprises a nucleotide sequence complementary to a polynucleotide containing the nucleotide sequence of the indicator gene or to the complementary strand thereof and that comprises at least 15 nucleotide. Alternatively, this invention relates to a reagent for testing for bronchial asthma, said reagent comprising an antibody that recognizes a peptide containing the amino acid sequence of the indicator protein. Oligonucleotides and antibodies composing the reagent of this invention may be appropriately labeled, or immobilized onto a suitable carrier according to the assay format. Further, the reagent of this invention may be combined with, as well as the oligonucleotides or antibodies as described above, additional elements necessary for the test or storage to form a kit. Additional elements that can be used for constituting a kit are shown below. These elements may be previously mixed as necessary, or added with preservatives and antiseptics:

buffer for diluting reagents and biological samples;

allergen to stimulate T-cells;

positive standard sample;

negative standard sample;

substrate for measuring labels;

reaction vessel; and

manual describing the assay protocol.

When PBMC was stimulated by an allergen in vitro, the expression level of the indicator gene in the present invention was showed to increase in T cells contained in the PBMC. Therefore, test for allergic diseases such as bronchial asthma and atopic dermatitis can be performed using the expression level of indicator gene as an index.

Test for an allergic disease in the present invention includes, for example, the tests as described below. Even a patient who, in spite of manifestation of bronchial asthma, can be hardly diagnosed with an allergic disease by conventional tests can be easily judged to be an allergic disease patient by carrying out the tests based on this invention. More specifically, the increase in the expression level of the indicator gene in a patient showing symptoms suspected of allergic disease indicates a high possibility that the symptoms are caused by an allergic disease. There are two types of bronchial asthma, one type being caused by an allergic reaction, and the other type not. Since treatments for two types are completely different, diagnosis as to which type causes the bronchial asthma is a very important step in the treatment. The test method of this invention can provide an extremely important information in identifying causes of bronchial asthma.

Alternatively, the present invention enables a test for judging whether the allergic symptom is getting ameliorated or not. The expression level of the indicator gene of the present invention indicated increase in T cells contained in PBMC upon stimulation of PBMC by an allergen in vitro. T cells are the cells playing a role as a headquarter in the immune response. Therefore, in PBMC stimulated by an allergen, a gene the expression level of which is varied in T cells that control its immune response is useful in judging the treatment effect. More specifically, an increase in the expression level of an indicator gene in a patient who has been diagnosed with an allergic disease indicates that allergic symptoms are highly likely in progress.

The present invention also relates to the use of transgenic non-human animals in which the expression level of an indicator gene in T cells has been elevated, as a model animal for an allergic disease. Allergic disease model animals are useful for clarifying in vivo changes in bronchial asthma. Furthermore, the allergic disease model animals of the present invention are useful in the assessment of therapeutic agents for the allergic bronchial asthma.

The present invention has revealed that the expression level of the indicator gene in T cells contained in PBMC is elevated upon stimulation of the PBMC with an allergen in vitro. Therefore, animals in which the expression levels of the MAL gene or genes functionally equivalent thereto in T cells have been artificially increased can be used as the allergic disease model animal. Increase in the expression level of an indicator gene in T cells includes that increase in the whole blood cells. In other words, the expression level of the indicator gene is increased not only in T cells but also in the whole blood cells or whole body.

A functionally equivalent gene in the present invention means a gene encoding a protein having a similar activity to that clarified in the protein encoded by the indicator gene. An example of functionally equivalent genes is a counterpart of the indicator gene in that animal species that is intrinsic to a transgenic animal.

Alternatively, a gene encoding a protein having, for example, 90% or more, preferably 95% or more, further preferably 99% or more homology to the amino acid sequence of the human MAL protein can be shown as a gene functionally equivalent to the MAL gene. In addition, a gene that can be amplified using oligonucleotides comprising the nucleotide sequence set forth in SEQ ID Nos: 1 and 2 used in Examples as the primers, and that the expression level thereof in T cells in PBMC is increased by stimulation with an allergen is also a gene functionally equivalent to the MAL gene.

A gene the expression level of which is increased in T cells contained in PBMC upon stimulation thereof by an allergen in vitro can be said to be involved in the pathway of allergic immune response. In other words, it is thought that stimulation by an allergen is transduced into allergic symptoms via the increase in the expression levels of these genes involved in the pathway. That is, a gene the expression level of which is increased in T cells contained in PBMC upon stimulation thereof with an allergen in vitro is said to be the gene that plays an important role in the allergic immune response in PBMC. Therefore, drugs that either suppress the expression of this gene or inhibit the activity thereof are expected to be active not only in ameliorating allergic symptoms but also removing the essential cause for developing allergic pathological conditions, in treatment of allergy.

As described above, a gene the expression level of which in T cells contained in PBMC is increased upon stimulation thereof with an allergen in vitro is very important. Therefore, it is highly significant to assess the role of the gene and effects of drugs targeting this gene using transgenic animals, which can be obtained by elevating the expression level of this gene in vivo, as the allergic disease model animal.

Allergic disease model animals according to the present invention are useful in not only screening for drugs for treating or preventing allergic diseases as described below but also elucidating mechanisms of allergic diseases, furthermore, testing the safety of compounds screened.

For example, if allergic disease model animals according to the present invention either develop clinical manifestations of bronchial asthma or show changes in measured values related to any allergic diseases, it is possible to construct a screening system for searching for a compound having activity to recover normal conditions.

In the present invention, increase in the expression level means the state wherein a target gene is transduced as a foreign gene and forcibly expressed; the state wherein transcription of a gene inherent in the host and translation thereof into protein are increased; or the state wherein decomposition of the translation product, protein, is suppressed. Gene expression level can be confirmed by, for example, the quantitative PCR as described in Examples. Furthermore, activity of translation product, protein, can be confirmed by comparing to that in the normal state.

A typical transgenic animal is the one to which a gene of interest is transduced to be forcibly expressed. Examples of another type of transgenic animals are those in which a mutation is introduced into the coding region of the gene to increase its activity or to modify the amino acid sequence of the gene product protein so as to be hardly decomposed. Examples of mutation in the amino acid sequence are the substitution, deletion, insertion, or addition of amino acid(s). In addition, by mutagenizing the transcriptional regulatory region of the gene, the expression itself of the gene of this invention can be controlled.

Methods for obtaining transgenic animals with a particular gene as a target are known. That is, a transgenic animal can be obtained by a method wherein the gene and ovum are mixed and treated with calcium phosphate; a method wherein the gene is introduced directly into the nucleus of oocyte in pronuclei with a micropipette under a phase contrast microscope (microinjection method, U.S. Pat. No. 4,873,191); or a method wherein embryonic stem cells (ES cells) are used. Furthermore, there have been developed a method for infecting ovum with a gene-inserted retrovirus vector, a sperm vector method for transducing a gene into ovum via sperm, or such. Sperm vector method is a gene recombination technique for introducing a foreign gene by fertilizing ovum with sperm after a foreign gene has been incorporated into sperm by the adhesion or electroporation method, etc. (M. Lavitranoet, et al. Cell, 57, 717, 1989).

Transgenic animals used as the allergic disease model animal of the present invention can be produced using all the vertebrates except for humans. More specifically, transgenic animals having various transgene and being modified gene expression levels thereof are produced using vertebrates such as mice, rats, rabbits, miniature pigs, goats, sheep, or cattle.

Furthermore, the present invention relates to a method of screening for a therapeutic agent for an allergic disease. In this invention, the indicator gene shows a significant increase in its expression level in T cells contained in PBMC when the PBMC is stimulated with an allergen in vitro. Therefore, it is possible to obtain a therapeutic agent for an allergic disease by selecting a compound capable of reducing the expression level of such a gene. Compounds that reduce the expression level of a gene are those having inhibitory effects on any steps of the transcription or translation of a gene, or the activity expression of a protein.

A method of screening for a therapeutic agent for an allergic disease of this invention can be carried out either in vivo or in vitro. This screening method can be carried out, for example, according to the steps as described below. The indicator gene in the screening method of this invention includes, in addition to the MAL gene, any genes functionally equivalent thereto. The steps of the screening method are:

(1) administering a candidate compound to a test animal;

(2) measuring the expression level of the indicator gene in a biological sample from the test animal; and

(3) selecting a compound that reduces the expression level of the indicator gene, compared to a control.

A functionally equivalent gene in the present invention means a gene encoding a protein having a similar activity to that clarified in the protein encoded by the indicator gene. An example of functionally equivalent genes is a counterpart of the indicator gene in that animal species that is intrinsic to a transgenic animal.

As a test animal in the screening method of the present invention, for example, an allergic disease model animal may be used. An allergic disease model animal is well known. For example, as a model closely resembling the human atopic dermatitis, there has been reported a model for the spontaneous dermatitis using NC/Nga mice. By administering the mite antigen (5 μg/ear) to the auricle of the mouse eight times in total at 2-3 days intervals, symptoms very similar to the human atopic dermatitis can be induced two weeks later. By administering a candidate compound to this system, and monitoring changes in the expression level of the indicator gene of this invention, the screening of this invention can be carried out.

By administering a drug candidate compound to a test animal as described above, and monitoring the action of the compound toward the expression of the indicator gene in the biological sample from the test animal, effects of drug candidate compounds on the expression level of indicator gene can be evaluated. Changes in the expression levels of the indicator gene in biological samples from test animals can be monitored by a method similar to the test method of this invention. Furthermore, based on the result of the evaluation, by selecting drug candidate compounds that reduce the expression level of the indicator gene, they can be screened.

More specifically, the screening according to the present invention can be carried out by comparing the expression level of the indicator gene in the biological sample collected from a test animal to that in a control. As a biological sample, the whole blood, PBMC, T cells, and such can be used. Methods for collecting and preparing these biological samples are known.

These screening methods enable the selection of drugs involved in the expression of indicator gene in various ways. More specifically, for example, drug candidate compounds having the following functions can be found:

suppression of signal transduction pathway to induce the expression of an indicator gene;

suppression of the transcription activity of the indicator gene; and

inhibition of stabilization or facilitation of decomposition of the transcription product of the indicator gene.

Furthermore, the present invention relates to a screening method comprising the step of stimulating a test animal with an allergen before and/or after the administration of a candidate compound in the screening method. In the case of stimulation with an allergen prior to the administration of a candidate compound, the activity of the candidate compound to suppress the immune response following the allergen stimulation can be detected. Compounds that can be obtained by such a screening are expected to have therapeutic effects on allergic diseases. On the other hand, in the case of allergen stimulation after the administration of a candidate compound, the activity of the candidate compound to suppress the onset of immune response triggered by the allergen stimulation can be detected. Compounds obtained by such a screening are expected to exert prophylactic effects for allergic diseases. Examples of allergens that can be used in the screening method of this invention are described above.

Examples of in vitro screening include a method in which cells expressing an indicator gene are contacted with a candidate compound to select a compound that reduces the expression level of the indicator gene. This screening may be carried out, for example, according to the steps of:

(1) contacting a candidate compound with cells expressing an indicator gene;

(2) measuring the expression level of the indicator gene; and

(3) selecting a compound that reduces the expression level of the candidate gene, compared to a control.

As cells in which an indicator gene expresses, for example, the human acute leukemia T cell strain Jurkat (ATCC No: TIB-152) is preferable for the screening method of the present invention. The cell strain is commercially available from ATCC.

In the screening method of this invention, first a candidate compound is added to the cell strain. Then, the expression level of an indicator gene in the cell strain is measured to select a compound that reduces the expression level of the gene.

In the screening method of this invention, expression levels of indicator genes can be compared not only based on the expression levels of proteins encoded by these genes but also based on the corresponding mRNAs detected. For performing the comparison of expression levels using mRNA, the process for preparing mRNA sample as described above is carried out in place of the process for preparing protein samples. Detection of mRNA and protein can be performed by known methods as described above.

Furthermore, based on the disclosure of this invention, it is possible to obtain the transcriptional regulatory region for the indicator gene of this invention and construct a reporter assay system. Reporter assay system means a system for screening for a transcriptional regulatory factor that acts on the transcriptional regulatory region using the expression level of a reporter gene localized downstream of the transcriptional regulatory region as an index.

The transcription activity of the approximately 600-bp region upstream from the transcription initiation point of the MAL gene has been studied using the luciferase gene as a reporter gene. As a result, the transcriptional factor recognition region such as SP-1 has been reported to play an important role in the transcription (Tugores, A. et al. 1997, DNA and Cell Biology 16, 245-255. A Tandem Array of Sp-1 and a Reverse Initiator Element Are Both Required for Synergistic Transcriptional Activation of the T-Cell-Specific MAL Gene). However, the transcriptional regulatory region used in the screening method of this invention is not limited to the 600 bp-region the activity of which has been revealed. If another transcriptional regulatory region for the MAL gene localized further upstream is clarified, the region will be also utilized in this invention. As described above, by constructing a reporter assay system using a transcriptional regulatory region having a sufficiently wide range, the screening of this invention can be carried out.

Transcriptional regulatory region is exemplified by promoter, enhancer, and furthermore, CAAT box, TATA box, and such, that are usually found in the promoter region. As a reporter gene, CAT (chloramphenicol acetyltransferase) gene, luciferase gene, growth hormone gene, and such can be utilized.

As already described, in the MAL gene, approximately 600-bp region upstream from the transcription initiation point has been analyzed as the transcriptional regulatory region. In addition, for example, the transcriptional regulatory region used in the screening of this invention can be also obtained as described below. That is, first, based on the nucleotide sequence of the indicator gene disclosed in this invention, the human genomic DNA library such as BAC library, YAC library, and such is screened by the methods using PCR or hybridization to obtain a genomic DNA clone containing the sequence of the cDNA. Based on the resulting genomic DNA sequence, the transcriptional regulatory region of the cDNA disclosed in this invention is predicted to obtain the transcriptional regulatory region. The transcriptional regulatory region obtained is cloned so as to be localized upstream of the reporter gene to prepare a reporter construct. The resulting reporter construct is introduced into a cultured cell strain to prepare a transformant for screening. By contacting the transformant with a candidate compound, a compound that controls the expression of the reporter gene can be screened for.

As an in vitro screening method according to this invention, a screening method based on the activity of the indicator protein can be also used. That is, the present invention relates to a method of screening for a therapeutic agent for an allergic disease, wherein the indicator gene is either the MAL gene or a gene functionally equivalent to MAL. The method comprises the steps of:

(1) contacting a candidate substance with a protein encoded by the indicator gene;

(2) measuring the activity of the protein; and

(3) selecting a compound that reduces the activity of the protein, compared to a control.

As for MAL, the indicator protein of this invention, for example, there have been assumed a possibility of its involvement in the T cell differentiation, transport of myelin protein to the myelin membrane, its relation to the delivery of various proteins including the GPI-anchor type protein to the apical membrane in the epithelial cell, or its relationship with CD59 and tyrosine kinase Lck. Using these activities as the index, it is possible to screen for compounds that reduce the activity of the MAL protein. Compounds that can be obtained as above suppress the activity of MAL. Thus, through the inhibition of an indictor protein the expression of which is induced in T cells, the allergic immune response can be controlled by the compounds.

Polynucleotide, antibody, cell strain, or model animal necessary for various screening methods according to this invention can be previously combined into a kit. More specifically, for example, a kit may be composed of a cell expressing an indicator gene and a reagent to measure the expression level of the indicator gene. As a reagent for measuring the expression level of an indicator gene, for example, a polynucleotide containing the nucleotide sequence of at least one indicator gene, or an at least 15-nucleotide-long oligonucleotides containing a nucleotide sequence complementary to the complementary strand thereof can be used. Alternatively, antibody that recognizes a peptide containing the amino acid sequence of at least one indicator protein may be used as a reagent. In these kits may be packaged a substrate compound used for the detection of the indicator, medium and vessel for cell culturing, positive and negative standard samples, and furthermore, a manual describing how to use the kit.

Candidate test compounds used in such screening include, in addition to compound preparations synthesized by existing chemical methods such as steroid derivatives and compound preparations synthesized by combinatorial chemistry, mixtures of multiple compounds such as extracts from animal or plant tissues, or microbial cultures, and their purified preparations, etc.

The compound selected by the screening method of this invention are useful as a therapeutic agent for an allergic disease. Also, the antisense DNA that can suppress the expression of the MAL gene, and, furthermore, antibody recognizing the protein encoded by the MAL gene are also useful as the therapeutic agent for an allergic disease. The therapeutic agent for an allergic disease according to this invention can be formulated by including the compound selected by the screening method as the effective ingredient, and mixing with a physiologically acceptable carrier, excipient, diluent, or the like. Aiming at the amelioration of allergic symptoms, the therapeutic agent for an allergic disease of this invention can be administered orally or parenterally.

Oral drugs can take any dosage forms selected from a group of granule, powder, tablet, capsule, solution, emulsion, suspension, etc. Injections can include the subcutaneous injection, intramuscular injection, intraperitoneal injection, etc.

Furthermore, for administering the compound that is composed of protein, the therapeutic effect can be achieved by introducing a gene encoding the protein into the living body using gene therapeutic techniques. The techniques for treating disease by introducing a gene encoding a therapeutically effective protein into the living body and expressing it therein are known.

Alternatively, the antisense DNA can be incorporated downstream of an appropriate promoter sequence to be administered as an antisense RNA expression vector. When this expression vector is introduced into T cells of an allergic disease patient, the therapeutic effect on allergic disease can be achieved by reducing the expression level of the gene through the expression of corresponding antisense gene. For introducing the expression vector into T cells, methods performed either in vivo or in vitro are known.

Although the dosage may vary depending on the age, sex, body weight, and symptoms of a patient, treatment effects, method for administration, treatment duration, type of active ingredient contained in the drug composition, or such, it can be usually administered in the range of 0.1 mg˜500 mg, preferably 0.5 mg˜20 mg per dose for an adult. However, since the dosage varies according to various conditions, amount less than the above-described dosage may be sufficient in some cases, and dosage exceeding the above-described range may be required in other cases.

The present invention provided the MAL gene, the expression level of which is significantly elevated in T cells contained in PBMC upon the stimulation thereof with an allergen in vitro. Based on the indicator gene of this invention, it has become possible to test for allergic diseases, and screen for therapeutic agents.

Expression levels of allergic disease-associated genes provided by the present invention can be easily detected regardless of types of allergens. Therefore, pathological conditions of allergic diseases can be comprehensively understood.

In addition, since the method for testing for allergic disease according to the present invention can analyze the expression level of genes using the peripheral blood leukocytes as a specimen, it is less invasive to patients. Furthermore, in the gene expression analysis, different from the protein measurement such as ECP, this method allows a highly sensitive measurement with a trace sample. The gene analysis technique has been more and more high-throughput and inexpensive year after year. Therefore, the test method according to this invention is expected to become an important bedside diagnostic method in near future. In this sense, these genes associated with pathological conditions are highly valuable in diagnosis.

Furthermore, the screening method of this invention is carried out using genes, the expression levels of which are significantly elevated in PBMC upon the stimulation thereof with an allergen in vitro as the indicator. T cells control the allergic immune response toward an allergen. Therefore, compounds which can be detected by this screening method are expected to be useful in controlling a wide range of allergic pathological conditions.

Any patents, patent applications, and publications cited herein are incorporated by reference.

The present invention is specifically illustrated below with reference to Examples, but it is not construed as being limited thereto.

### EXAMPLE 1

### Blood Sample Collection from Patients and Normal Healthy Subjects

For isolating a gene the expression level of which changes specifically to allergic disease, blood samples were collected from normal healthy subjects and patients selected by analyzing the cases of the disease. Blood samples were collected from 18 normal healthy subjects, a group of 8 patients with bronchial asthma, a group of 6 patients with atopic dermatitis, and 5 patients with both bronchial asthma and atopic dermatitis. Parts of the blood samples were used for measuring mite antigen-specific IgE level.

For the specific IgE measurement, the CAP RAST method was employed, which is an improved RAST method that uses a paper disk as a solid phase. Serum (Pharmacia) with standard antibody titer was used as the standard to determine the IgE antibody titer in respective samples, and the results were expressed as scores.

Scores of the mite-specific IgE antibody titer from each subject are shown in Table 2. As shown in the table, the scores of not less than a half of the normal healthy subjects were 2 or less, while high scores were observed in the patient groups, indicating that these patients have an allergy toward the mite antigen.

### EXAMPLE 2

### Preparation of Lymphocyte Fractions from Blood Samples

PBMC were prepared from 10 ml blood sample as follows. First, 1 ml heparin (purchased from Novo Co., etc.) was spread over the 10 ml-syringe wall surface, and then 10 ml blood sample including a final concentration of 50 units/ml heparin was taken. For blood collection, two 22G needles for each person were prepared. After removing the needle from the syringe, the blood sample was transferred to a 50-ml centrifuge tube (polypropylene). The tube was centrifuged at 1500 rpm for 5 min at room temperature and then 1.1 ml was taken from as close to the surface as possible. After further 15000 rpm centrifugation for 5 min at 4° C., 1 ml of the supernatant was collected as plasma. An equal amount (9 ml) of 0.9% NaCl containing 3% dextran (Nacalai) was added to the remaining sample. This mixture was inverted gently several times, and then let stand for 30 min at room temperature. PRP (platelet rich plasma) was transferred to a new 15 ml centrifuge tube and centrifuged at 1200 rpm (equivalent to 150×g for the Tomy centrifuge) for 5 min at room temperature. After the centrifugation, platelets were present in the supernatant. Precipitated cells were resuspended in 5 ml Ca and Mg-free HBSS (GIBCO, etc.). The cell suspension was layered on the top of a 5 ml Ficoll Paque (Pharmacia)-containing Falcon tube (2006 or 2059, polypropylene) by use of a capillary pipette. After centrifuging the tube at 1200 rpm for 5 min, it was further centrifuged at 1500 rpm (equivalent to 400×g for the Tomy centrifuge) for 30 min at room temperature. As a result, granulocytes and erythrocytes were precipitated, and lymphocytes, monocytes, and platelets were included in the middle layer, with the Ficoll layer between the precipitate and the middle layer. The middle layer was separated as PBMC.

### EXAMPLE 3

### Gene Expression Analysis of T Cells in PBMC Stimulated with Mite-Specific Antigen Using GeneChip

Mite antigen is one of the typical allergens. Aiming at discovering an allergic disease-associated gene that is likely responsive to the stimulation with the mite antigen, PBMCs derived from patients with allergic diseases and those derived from normal healthy subjects were stimulated with the mite protein antigen separately to search for genes which show expression variations different between both of them.

From 100 ml each of peripheral blood samples collected from each one of patients with an allergic chronic bronchial asthma and normal healthy subjects, PBMCs were prepared according to the method described in Example 2. Resulting PBMCs were cultured under the three different conditions, namely, in the presence of mite extracts (5 μg/ml each of Mite Extract Df and Mite Extract Dp, LSL Co.), in the presence of mite extracts and dexamethasone (100 nM), and with no addition (control). Rosweli Park Memorial Institute (RPMI) 1649 medium containing 10% fetal bovine serum, penicillin (100 U/ml), and streptomycin (100 μg/ml) was used. After the 12-h or 40-h cultures, PBMC was collected to separate T cells (CD3-positive cells) and monocytes (CD14-positive cells) contained therein. T cells and monocytes were successively labeled with the anti-CD3 antibody magnetic beads and anti-CD14 antibody magnetic beads, respectively, and separated using the magnetic cell-sorter (Milteni, Biotech Co.). RNA was extracted from the resulting cells, and analyzed for the gene expression using the oligonucleotide array (GeneChip HuGene FL Array (Affymetrix)). Furthermore, IL-4 contained in the culture supernatant recovered at that time was measured by ELISA (Example 5).

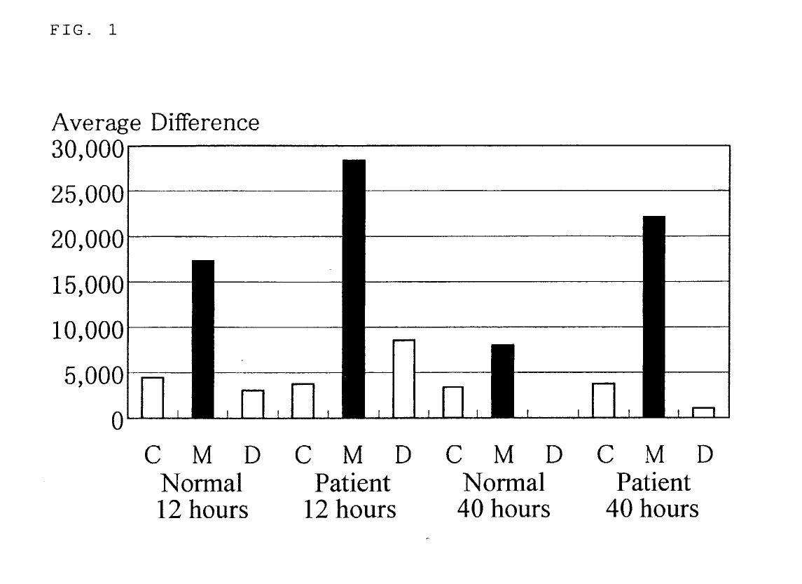

As a result, it was revealed that the expression of the X76223 gene was elevated in T cells from the asthma patients 12 and 40 h after the antigen stimulation compared with those from normal healthy subjects (FIG. 1). Furthermore, the increase in the MAL expression was suppressed by the action of a steroidal therapeutic agent, dexamethasone. X76223 was registered on the oligonucleotide array as the Homo sapiens MAL gene exon 4.

### EXAMPLE 4

### Quantitative Analysis of MAL Expression in PBMC Stimulated with Mite Antigen

To confirm the results of DNA chip experiments, PBMC prepared from the peripheral blood of subjects shown in Table 2 was stimulated with the mite antigen to measure the MAL expression level using the quantitative RT-PCR method. After the stimulation by the same method as described above, RNA was prepared from the recovered whole PBMC, and the culture supernatant was stored. After cDNA was prepared from the RNA by the standard method, the quantitative RT-PCR was carried out for MAL and IL-4 receptor α chain using the respective specific primers and probes with the ABI7700 (Applied Biosystems). Those primers and probes were designed based on the MAL sequence registered on the chip.

MAL Primers

X76223-f: AAAAGCCCTGCCCTGTTGCT (SEQ ID NO: 1)

X76223-r: CCCCGAACAAGAAGGTCCCC (SEQ ID NO: 2)

MAL Probe

X76223p: TGCTGTGTTTACTCTCCCGTGTGCC (SEQ ID NO: 3)

IL-4 Receptor α Chain Primer

X52425-f: CGACTTGTGAACGAGTTGTTGG (SEQ ID NO: 4)

X52425-r: TTCAGTGAGACAGAGGCAGGTG (SEQ ID NO: 5)

IL-4 Receptor α Chain Probe

X52425p: TGTTGTAACTGCCCAAGGCATGTTTTGC (SEQ ID NO: 6)

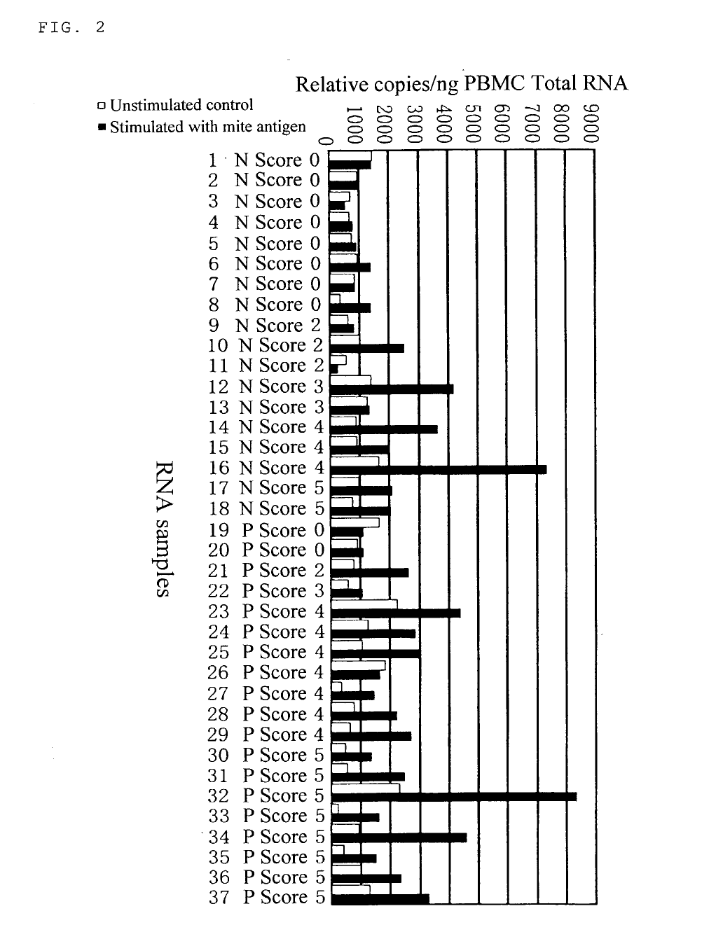

Probes for quantitating MAL or IL-4 receptor α chain are both labeled with FAM (6-carboxy-fluorescein) at their 5′-ends, and with TAMRA (6-carboxy-N,N,N′,N′-tetramethylrhodamine) at their 3′-ends. As for MAL, a plasmid, into which a 151-bp sequence to be amplified with the primers had been cloned, was used as a standard for the copy number. As for the IL-4 receptor α chain, a plasmid into which a 135-bp sequence to be amplified with the primers had been cloned was used as a standard for the copy number. The composition of the reaction solution for monitoring PCR amplification is shown in Table 1, and the results of quantitation are shown in Table 2. Changes in the MAL expression levels depending on the presence or absence of the mite antigen stimulation are represented in FIG. 2.

TABLE 1
Composition of reaction solution for ABI-PRISM 7700 (volume per
well)

| | | |
|---|---|---|
| Sterile distilled water | 23.75 | (μl) |
| 10x TaqMan buffer A | 5 | |
| 25 mM MgCl₂ | 7 | |
| dATP (10 mM) | 1.0 | |
| dCTP (10 mM) | 1.0 | |
| dGTP (10 mM) | 1.0 | |
| dUTP (10 mM) | 1.0 | |
| Forward Primer (10 μM) | 1 | |
| Reverse Primer (10 μM) | 1 | |
| TaqMan Probe (2 μM) | 2.5 | |
| AmpliTaq Gold (5 U/μl) | 0.25 | |
| AmpErase UNG (1 U/μl) | 0.5 | |
| Template solution | 5 | |
| Total | 50 | |

TABLE 2

| | | | | | | IL-4 receptor α | | IL-4 in culture | |
|---|---|---|---|---|---|---|---|---|---|
| | | | Total | MAL expression level | | expression level | | supernatant | |
| Sub- | | Mite-specific | IgE | (copy number/ng RNA) | | (copy number/ng RNA) | | (ρg/ml) | |
| ject | Disease | IgE class | (IU/ml) | Unstimulated | Mite stimulation | Unstimulated | Mite stimulation | Unstimulated | Mite stimulation |
| 1 | normal | 0 | 21 | 1451.4 | 1410.0 | 2152.3 | 2526.9 | 0.098 | 0.143 |
| 2 | normal | 0 | 61 | 941.6 | 941.4 | 1796.0 | 1910.0 | 0.271 | 0.393 |
| 3 | normal | 0 | 63 | 716.2 | 514.5 | 1869.9 | 1774.3 | 0.179 | 0.304 |
| 4 | normal | 0 | 170 | 658.7 | 759.6 | 1290.5 | 1644.7 | 0.150 | 0.470 |
| 5 | normal | 0 | 20 | 743.3 | 898.6 | 1660.7 | 2107.9 | 0.123 | 0.261 |
| 6 | normal | 0 | 82 | 923.9 | 1368.0 | 1864.2 | 2526.6 | 0.027 | 0.414 |
| 7 | normal | 0 | 290 | 837.2 | 848.6 | 1627.1 | 1698.3 | 0.078 | 0.033 |
| 8 | normal | 0 | 20 | 355.2 | 1365.1 | 1361.1 | 2596.0 | 0.055 | 1.016 |
| 9 | normal | 2 | 460 | 604.8 | 790.7 | 1300.0 | 1955.0 | 0.189 | 0.135 |
| 10 | normal | 2 | 150 | 998.6 | 2511.6 | 1355.9 | 3480.4 | 0.000 | 0.426 |
| 11 | normal | 2 | 30 | 539.9 | 252.4 | 1695.4 | 1160.4 | 0.000 | 0.261 |
| 12 | normal | 3 | 110 | 1363.1 | 4178.7 | 2392.4 | 5717.9 | 0.019 | 1.737 |
| 13 | normal | 3 | 630 | 1242.6 | 1300.4 | 2159.4 | 1821.1 | 0.112 | 0.242 |
| 14 | normal | 4 | 170 | 855.7 | 3604.7 | 1318.6 | 5466.6 | 0.046 | 3.226 |
| 15 | normal | 4 | 530 | 887.6 | 1958.9 | 2257.8 | 3845.2 | 0.000 | 0.755 |
| 16 | normal | 4 | 390 | 1617.7 | 7331.2 | 2903.6 | 8268.0 | 0.437 | 2.130 |
| 17 | normal | 5 | 970 | 965.3 | 2082.8 | 2615.7 | 3725.1 | 0.044 | 2.131 |
| 18 | normal | 5 | 470 | 722.8 | 2008.5 | 3263.0 | 5206.7 | 0.060 | 1.500 |
| 19 | dermatitis | 0 | 140 | 1620.8 | 1087.2 | 2447.0 | 1815.4 | 0.000 | 0.067 |
| 20 | asthma | 0 | 140 | 885.3 | 1101.8 | 1478.1 | 1631.4 | 0.000 | 0.330 |
| 21 | asthma | 2 | 52 | 774.8 | 2610.8 | 1319.8 | 4020.8 | 0.000 | 1.528 |
| 22 | asthma | 3 | 90 | 563.9 | 1069.0 | 2644.9 | 3341.2 | 0.009 | 1.363 |
| 23 | dermatitis | 4 | 190 | 2255.5 | 4395.8 | 3388.2 | 4507.1 | 0.000 | 3.752 |
| 24 | dermatitis | 4 | 97 | 1235.2 | 2859.0 | 2200.0 | 4887.4 | 0.000 | 0.620 |
| 25 | dermatitis, asthma | 4 | 130 | 1055.6 | 2985.4 | 2022.0 | 3875.9 | 0.194 | 1.203 |
| 26 | dermatitis, asthma | 4 | 530 | 1829.3 | 1646.7 | 2564.6 | 2378.0 | 0.000 | 0.320 |
| 27 | asthma | 4 | 260 | 357.9 | 1445.1 | 1329.6 | 2885.0 | 0.000 | 1.036 |
| 28 | asthma | 4 | 180 | 759.0 | 2197.0 | 1581.2 | 2850.9 | 0.019 | 2.014 |
| 29 | asthma | 4 | 310 | 632.5 | 2703.0 | 1419.8 | 4150.6 | 0.027 | 2.098 |
| 30 | dermatitis | 5 | 9800 | 496.3 | 1348.6 | 1885.0 | 3342.8 | 0.000 | 0.632 |
| 31 | dermatitis | 5 | 18000 | 537.8 | 2470.6 | 1216.4 | 4524.7 | 0.000 | 2.165 |
| 32 | dermatitis | 5 | 1000 | 2301.4 | 8343.2 | 1985.3 | 9960.7 | 0.000 | 1.176 |
| 33 | dermatitis, asthma | 5 | 1100 | 232.5 | 1599.3 | 1336.0 | 2624.5 | 0.000 | 0.696 |
| 34 | dermatitis, asthma | 5 | 620 | 915.7 | 4574.6 | 1604.2 | 5607.6 | 0.203 | 1.742 |
| 35 | dermatitis, asthma | 5 | 1600 | 420.4 | 1489.8 | 2103.6 | 2644.7 | 0.002 | 9.307 |
| 36 | asthma | 5 | 460 | 1005.8 | 2346.9 | 3002.2 | 4278.9 | 0.044 | 1.346 |
| 37 | asthma | 5 | 860 | 1274.3 | 3298.8 | 2656.1 | 5502.1 | 0.000 | 1.023 |

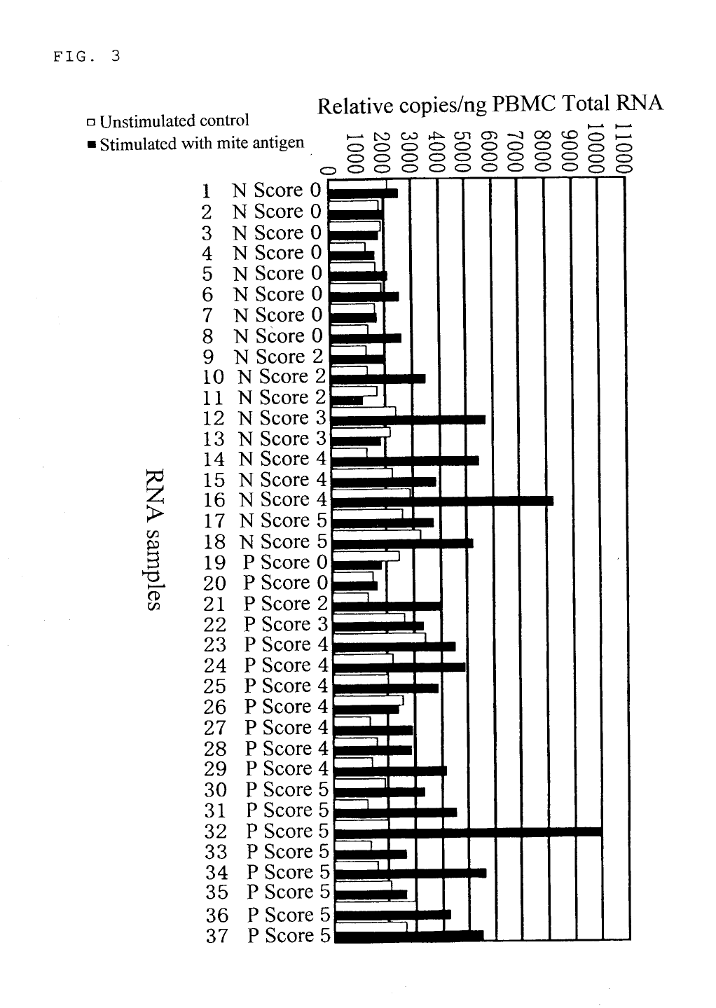

It was confirmed that the MAL expression was specifically elevated when PBMC was stimulated with the mite antigen, in samples derived from specific mite IgE positive subjects. A majority of patients are positive for the specific mite IgE antibody. As a result of repeated measure analysis of variance separately performed in the specific mite IgE positive and negative groups, a significant difference (p=0.0019) was found between them. The expression pattern of the IL-4 receptor α chain was very similar to that of the MAL expression (FIG. 3). As a result of repeated measure analysis of variance, a significant difference (p=0.0040) was also found between the specific mite IgE negative and positive groups. Expression variances of both of the genes showed an extremely high correlation (correlation coefficient=0.95).

### EXAMPLE 5

### Production of IL-4 by PBMC Stimulated with Mite Antigen

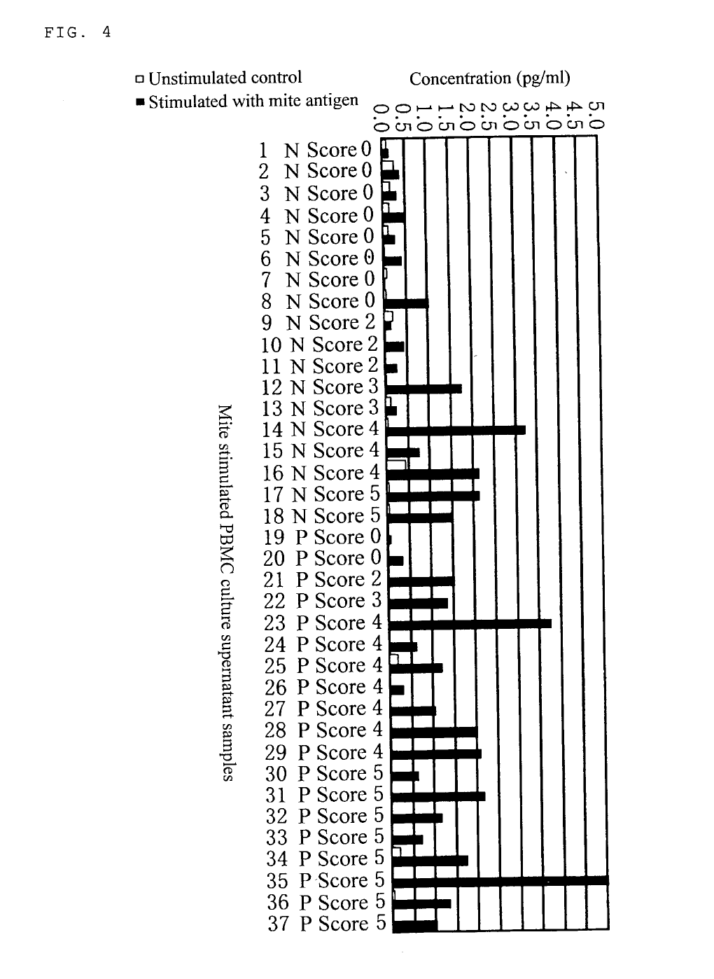

The amount of IL-4 that was produced and secreted into the culture supernatant was measured by ELISA (R & D System). In almost all cases of mite specific IgE positive subjects, the IL-4 production was found when being stimulated with the antigen. At the same time, the MAL expression was also increased in these samples (FIG. 4).

### EXAMPLE 6

### Quantification of MAL Expression in Various Types of Leukocytes

MAL expression was quantitated in T cells, B cells, monocytes, neutrophils, and eosinophils prepared from the peripheral blood samples from 5 normal healthy subjects. To the whole blood collected from the subjects was added 3% dextran solution, and the mixture was allowed to stand at room temperature for 30 min to sediment erythrocytes. The leukocyte fraction in the upper layer was collected, layered on a Ficoll solution (Ficoll-Paque PLUS; Amersham Pharmacia Biotech), and centrifuged at 1500 rpm for 30 min at room temperature. The granulocyte fraction recovered in the lower layer was reacted with the CD16 antibody magnetic beads at 4° C. for 30 min, and cells that were not trapped and eluted in the separation step using MACS were used as the eosinophils in experiments. After the elution of eosinophils, neutrophils (N) were prepared by releasing the cells, which were trapped with CD16 antibody magnetic beads, from the magnetic field, eluting, and recovering. On the other hand, the monocyte fraction recovered in the middle layer by the Ficoll-centrifugation was separated into the fraction eluted from MACS CD3 antibody magnetic beads (mixture of M (monocyte) and B cell) and fraction trapped therein (T-cell fraction). Then, using MACS CD14 antibody magnetic beads, the eluted fraction was separated into the eluted fraction (B cell fraction) and trapped fraction (monocyte fraction), and those three fractions were referred to as the purified T cells, B cells, and monocytes.

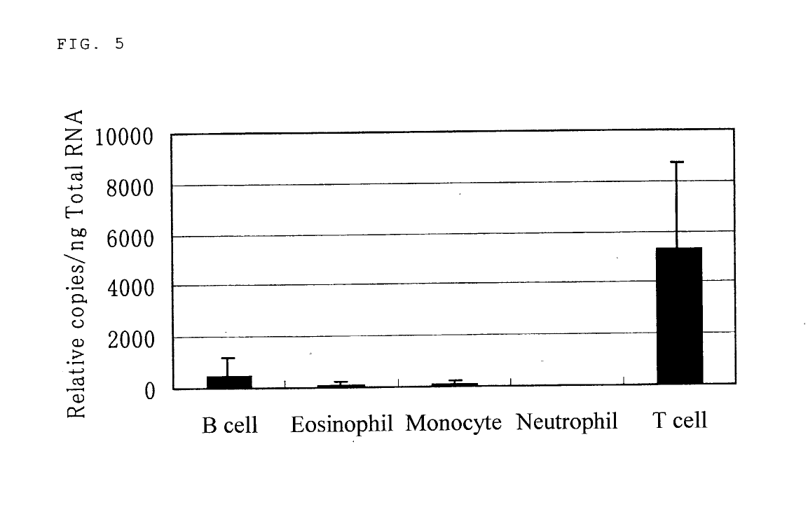

Eosinophils were solubilized using Isogen, while neutrophils, T cells, B cells and monocytes were solubilized with RNeasy (Qiagen), and total RNA were extracted, treated with DNase (by the same methods as described above), and subjected to the gene expression analysis. Primers, probes, and so forth used were the same as above. The measurement results are shown in FIG. 5. These results clearly showed a high level of MAL expression in T cells in particular.

### EXAMPLE 7

### Expression Induction Experiment with IL-4 in Cultured T Cells Derived from Peripheral Blood

Effects of various cytokines on the MAL expression level were examined. T cells were separated from the peripheral blood sample from an normal healthy subject using the method described in Example 6, and stimulated with the anti-CD3 antibody to induce the cell growth. That is, on a culture plate which was surface-treated with the anti-CD3 antibody, the separated T cells were cultured in a 5% FCS-containing RPMI1640 medium (1 mM sodium pyruvate, 2 mM L-glutamine, 10 U/ml penicillin, 100 μg/ml streptomycin, and 200 U/ml IL-2) at a density of 5×10⁵ cells/ml for 5 days. Grown T cells were diluted with the same medium to a density of 5×10⁵ cells/ml, and cultured on a non-surface treated plate for further 3 days. The resulting cells were washed with the medium without IL-2, suspended in the medium (without IL-2) supplemented with cytokine for the stimulation at a density of 1×10⁶ cells/ml, and cultured under the stimulation for a predetermined period of time. Cells were then collected to prepare RNA. Cytokines used are shown below:

IL-4: 2 ng/ml;

IL-12: 2 ng/ml;

IFN-γ: 200 ng/ml; and

IFN-α: 100 IU/ml.

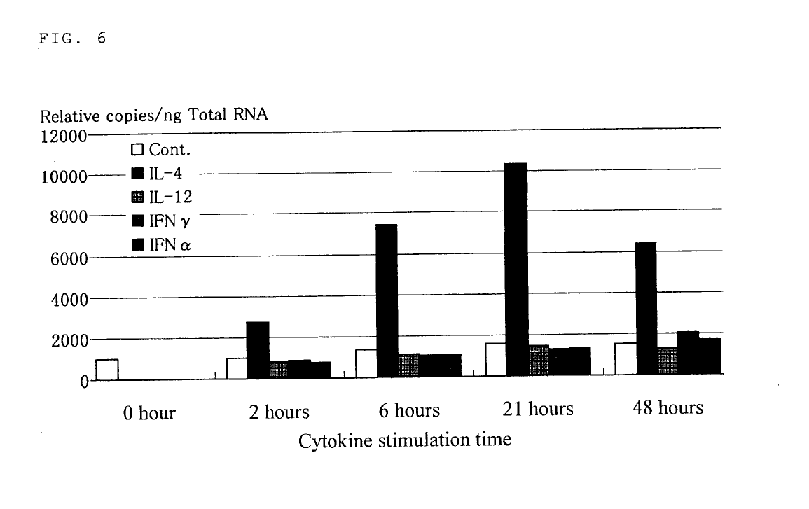

The same medium as that described in Example 3 was used. During the cultivation for 0˜48 h, mRNA for MAL was induced in the case of stimulation with IL-4. No such action was observed with IFN-γ, IFN-α, or IL-12 (FIG. 6). Induction of MAL expression with IL-4 may indicate the association of MAL with the action of IL-4 in allergic diseases. For example, MAL may act during the differentiation process of T cell into Th2, and also may associate with the production of Th2 cytokines, IL-4, IL-5, and IL-10.

### EXAMPLE 8

### Induction of MAL Protein Expression with IL-4 in Jurkat Cell Line

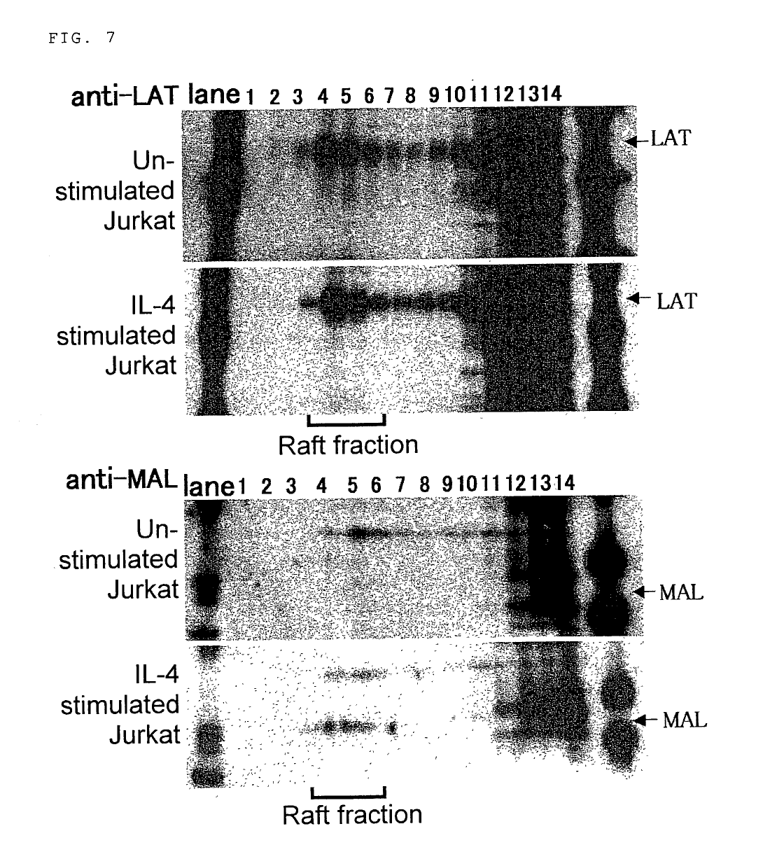

Cell lysate was prepared from Jurkat cells that had been stimulated with IL-4 (2 ng/ml) for 32 hours, and subjected to sucrose density gradient centrifugation according to the method of Fernando Martin-Belmonte et al. (Fernando Martin-Belmonte, Rosa Puertollano, Jaime Millan, and Miguel A. Alonso. The MAL Proteolipid Is Necessary for the Overall Apical Delivery of Membrane Protein in the Polarized Epithelial Madin-Darby Canine Kidney and Fischer Rat Thyroid Cell Lines. Molecular Biology of the Cell 11, 2033-2045, June 2000) to separate the raft fraction (fractions 4, 5, and 6). Proteins in each fraction were separated by SDS polyacrylamide gel electrophoresis, and analyzed by Western blotting using antibody against MAL peptide or that against LAT (linker for activation of T cells). LAT is a protein that has been proved to exist in the raft so as to be able to serve as a marker of the raft fraction. As a result, it was found that the expression of MAL protein is increased in the raft (FIG. 7).

### EXAMPLE 9

### Induction of MAL Protein Expression with IL-4 in Human Peripheral Blood T-Cells

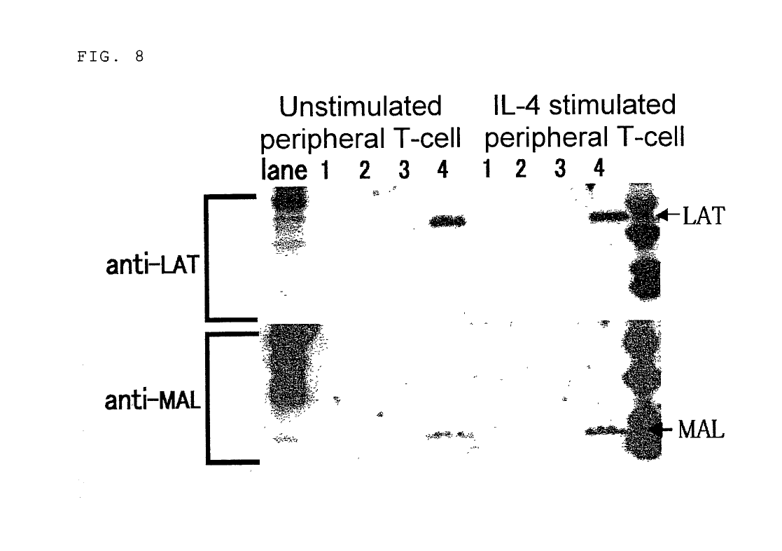

Human peripheral blood cells that had been cultured and propagated were similarly stimulated as in Example 7 with IL-4 (2 ng/ml) for 41 hours, and then the MAL protein expression in the raft fraction (fraction 4) was examined by Western blotting. As a result, the induction of the MAL protein expression by IL-4 was confirmed also in the raft of human peripheral blood T-cells (FIG. 8).

From the results described above, the induction of MAL expression by IL-4 was proved not only on the mRNA level but also the protein level in Jurkat cell line, as well as in human peripheral blood T cells. This confirmation of the increase in MAL protein in the raft, which is an important component for the signal transduction, indicated that MAL protein has an important function in the raft of T-cells.

What is claimed is:

1. A method for testing for an allergic disease using MAL as an indicator gene, said method comprising the steps of:
(a) measuring the expression level of the indicator gene in a biological sample from a subject, and
(b) comparing the expression level measured in (a) with that in a biological sample from a normal healthy subject.

2. The method according to claim 1, wherein the allergic disease is atopic dermatitis and/or bronchial asthma.

3. The method according to claim 1, wherein the expression level of the gene is measured by PCR of the cDNA for the gene.

4. The method according to claim 1, wherein the expression level of the gene is measured by detecting a protein encoded by said gene.

5. The method according to claim 1, wherein the biological sample is a sample comprising peripheral blood T cells.

6. The method according to claim 1, wherein the expression level of the indicator gene is measured after peripheral blood mononuclear cells (PBMC) are stimulated with an allergen.

7. A reagent for testing for allergic disease, said reagent comprising an oligonucleotide that comprises a nucleotide sequence complementary to a polynucleotide comprising the nucleotide sequence of MAL gene or to the complementary strand thereof and that comprises at least 15 nucleotides.

8. A reagent for testing for allergic disease, said reagent comprising an antibody that recognizes a peptide comprising the amino acid sequence encoded by MAL gene.

9. The reagent according to claim 7 or 8, said reagent further comprising an allergen.

10. A method of screening for a therapeutic agent for an allergic disease using MAL gene or a gene functionally equivalent thereto as an indicator gene, said method comprising the steps of:
(a) contacting a candidate compound with a cell expressing the indicator gene,
(b) measuring the expression level of the indicator gene, and
(c) selecting a compound that reduces the expression level of the indicator gene, compared to a control.

11. The method according to claim 10, wherein the cell is T cell.

12. A method of screening for a therapeutic agent for an allergic disease using MAL gene or a gene functionally equivalent thereto as an indicator gene, said method comprising the steps of:
(a) administering a candidate compound to a test animal,
(b) measuring the expression level of the indicator gene in a biological sample from the test animal, and
(c) selecting a compound that reduces the expression level of the indicator gene, compared to a control.

13. The method according to claim 12, said method comprising the step of stimulating the test animal with an allergen before or after step (a).

14. A method of screening for a therapeutic agent for an allergic disease using MAL gene or a gene functionally equivalent thereto as an indicator gene, said method comprising the steps of:
(a) contacting a candidate compound with a cell into which a vector comprising the transcriptional regulatory region of the indicator gene and a reporter gene that is expressed under the control of the transcriptional regulatory region has been introduced,
(b) measuring the activity of said reporter gene, and
(c) selecting a compound that reduces the expression level of said reporter gene, compared to a control.

15. A method of screening for a therapeutic agent for an allergic disease using MAL gene or a gene functionally equivalent thereto as an indicator gene, said method comprising the steps of:
(a) contacting a candidate compound with a protein encoded by the indicator gene,
(b) measuring the activity of said protein, and
(c) selecting a compound that reduces the activity of said protein, compared to a control.

16. A therapeutic agent for an allergic disease, said agent comprising, as an active ingredient, a compound that is obtained by the method according to any one of claims 10, 12, 14, and 15.

17. A therapeutic agent for an allergic disease, said agent comprising an antisense DNA against an indicator gene or a portion thereof as a principal ingredient, wherein the indicator gene is MAL gene or a gene functionally equivalent to MAL gene.

18. A therapeutic agent for an allergic disease, said agent comprising, as a principal ingredient, an antibody that binds to a protein encoded by an indicator gene, wherein the indicator gene is MAL gene or a gene functionally equivalent to MAL gene.

19. A method for producing an allergic disease model animal using MAL gene or a gene functionally equivalent thereto as an indicator gene, said method comprising a step of elevating expression level of the indicator gene in T cells of a non-human vertebrate.

20. A kit for screening for a therapeutic agent for an allergic disease, said kit comprising an oligonucleotide that comprises a nucleotide sequence complementary to a polynucleotide comprising a nucleotide sequence of an indicator gene or to the complementary strand thereof and that comprises at least 15 nucleotides and cells expressing the indicator gene, wherein the indicator gene is the MAL gene or a gene functionally equivalent to MAL gene.

21. A kit for screening for a therapeutic agent for an allergic disease, said kit comprising an antibody that recognizes a peptide comprising an amino acid sequence encoded by an indicator gene and cells expressing the indicator gene, wherein the indicator gene is MAL gene or a gene functionally equivalent to MAL gene.

22. The kit according to claim 20 or 21, said kit further comprising an allergen.

23. A method for treating an allergic disease, said method comprising the step of administering a compound that is obtained by the method according to any one of claims 10, 12, 14, and 15.

24. A method for treating an allergic disease, said method comprising the step of administering an antisense DNA against MAL gene, a gene functionally equivalent to MAL gene, or a portion thereof.

25. A method for treating an allergic disease, said method comprising the step of administering an antibody that binds to a protein encoded by MAL gene.
